(19) United States
(12) Patent Application Publication
(10) Pub. No.: US 2026/0357974 A1
(43) Pub. Date: Nov. 12, 2026

(54) INTELLIGENT LIGHTING SYSTEM

(71) Applicant: Operative Insights, LLC, Chicago, IL (US)

(72) Inventors: Raheem Dajian Bell, Chicago, IL (US); Leonard Matthew Dunovant, IV, Chicago, IL (US); Ngoc Bao Tran, Chicago, IL (US); Hoang Anh Thu Nguyen, Chicago, IL (US)

(21) Appl. No.: 19/670,769

(22) Filed: May 7, 2026

Related U.S. Application Data
(60) Provisional application No. 63/804,578, filed on May 12, 2025.

(51) Int. Cl. F21V 21/15; F21V 21/28; F21V 23/04

(52) U.S. Cl. CPC F21V 21/15; F21V 21/28; F21V 23/0478

(57) ABSTRACT

A lighting system includes a support assembly, a lighting assembly movably coupled to the support assembly, the lighting assembly including one or more light sources and one or more sensors configured to generate sensor data associated with an environment, and a control system operatively coupled to the support assembly and the lighting assembly, the control system being configured to receive the sensor data from the one or more sensors, determine, based on the sensor data, a location of activity or a region of interest within the environment, and automatically control at least one of the support assembly or the lighting assembly to adjust at least one of a position of the lighting assembly, an orientation of the lighting assembly, or an illumination characteristic of the one or more light sources such that illumination is directed in response to the location of activity or the region of interest.

### CROSS-REFERENCE TO RELATED APPLICATION

This application claims the benefit of priority under 35 U.S.C. § 119(e) of U.S. Provisional Application No. 63/804,578, filed May 12, 2025, the contents of which are incorporated herein by reference in their entirety.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIG. 1 illustrates an example intelligent lighting system in a mobile floor-supported configuration according to some embodiments.

FIG. 2 illustrates an intelligent lighting system in a mounted configuration according to some embodiments.

FIG. 3 illustrates an example partitioned procedural environment in which the procedural field is divided into quadrants according to some embodiments.

FIG. 4 illustrates an intelligent lighting system in a mobile floor-supported configuration according to further embodiments.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

### DETAILED DESCRIPTION OF EXEMPLARY EMBODIMENTS

Surgical and other procedural environments often rely on lighting systems to provide localized illumination for a clinician or other user. Where such lighting systems may be used in operating rooms, related medical settings, and other environments requiring added illumination, procedural settings can present particular challenges due to the need for accurate, stable, and responsive lighting throughout the course of a procedure.

In many existing environments, lighting equipment is manually positioned or adjusted by a surgeon, nurse, assistant, or other staff member to direct light toward a region of interest. In some circumstances, such manual interaction with the lighting equipment can be undesirable. For example, contact with the lighting system during a procedure may interrupt workflow, may require additional assistance from personnel, and may create concerns associated with maintaining sterility in the procedural environment. Further, if illumination is not properly directed at a needed location, visibility may be reduced at times when prompt response is important.

Accordingly, it would be advantageous to provide improved lighting systems and related methods that can provide responsive illumination in a procedural environment while reducing reliance on manual adjustment. It would also be advantageous to provide lighting systems that can better adapt to user movement, changing procedural conditions, and different workflow demands within the environment.

The present disclosure is directed generally to an intelligent lighting system and related methods for providing adaptive illumination within an environment, such as a surgical or other procedural environment. As used herein, an intelligent lighting system (ILS) may refer to a lighting system configured to sense conditions within the environment and to responsively adjust one or more illumination characteristics based on detected inputs, learned patterns, or user commands.

In some embodiments, the intelligent lighting system may further be configured to obtain image data, spectral data, thermal data, or other sensed data from a target region and to process such data to perform one or more diagnostic functions. For example, the intelligent lighting system may be configured to assess tissue characteristics, detect abnormalities, identify regions of interest, monitor procedural conditions, or otherwise generate diagnostic information based on light reflected, emitted, or otherwise received from the target region. In this manner, the intelligent lighting system may serve not only as an illumination platform, but also as a sensing and analysis platform capable of supporting clinical evaluation, procedural guidance, or other diagnostic operations.

Although various embodiments are described herein in the context of medical, surgical, or other procedural environments, the disclosed intelligent lighting system (ILS) is not limited to such applications. In some embodiments, the intelligent lighting system may be used in any environment in which adaptive illumination, sensing, positioning, monitoring, imaging, or diagnostic functionality is desirable. By way of example, and not limitation, the disclosed systems and methods may be employed in industrial maintenance settings, construction sites, inspection operations, laboratory environments, workshop or hobby environments, educational settings, manufacturing facilities, automotive repair environments, or other commercial, residential, or technical applications. Accordingly, references herein to surgical, clinical, or procedural use are provided for illustration and should not be construed as limiting the scope of the disclosed systems, devices, or methods.

In some embodiments, the disclosed intelligent lighting system (ILS) may be implemented in a mobile floor-supported configuration in which one or more lighting assemblies, sensing components, control electronics, and power components are supported by a movable and free-standing base structure. The movable base structure may include a wheeled support assembly configured to permit repositioning of the system within a room, facility, or work area, thereby allowing the lighting system to be selectively deployed at different locations as needed.

The movable base structure may further support a support assembly including one or more articulated arms, adjustable mounts, or other positioning structures, and may further support display components, user input devices, imaging devices, and communication modules that cooperate to provide adaptive illumination and related sensing functions.

In further embodiments, the disclosed intelligent lighting system (ILS) may be implemented in a mounted configuration in which one or more support structures are secured to or suspended from a ceiling, overhead frame, ceiling track, mounting plate, wall or other elevated support. In some embodiments, the mounted configuration may include a support assembly extending from the elevated support and configured to position one or more lighting assemblies above a procedural surface or other target area.

The mounted configuration may further include one or more sensing components, control electronics, and power components supported by the elevated support, the support assembly, the lighting assembly, or combinations thereof. The support assembly may include one or more rigid members, articulated members, pivoting couplings, translational components, or other positioning structures that permit selective placement of a lighting assembly relative to the target area.

In some embodiments, the support assembly may include an actuatable joint configured to receive and support a lighting assembly and to permit controlled rotational, angular, translational, vertical, lateral, or other positioning thereof relative to a procedural surface or other target area. In some embodiments, the lighting assembly may be supported above a procedural surface and may include a plurality of light sources arranged such that the lighting assembly extends about, surrounds, or partially encircles the procedural surface from an elevated position.

As used herein, the term “lighting assembly” may refer to a lighting structure that includes one or more light sources and, in some embodiments, one or more supporting, mounting, actuation, optical, electrical, or control components associated with producing, directing, shaping, or adjusting emitted light. In some embodiments, the lighting assembly may include a housing, frame, or other support structure that carries the one or more light sources, and may further include one or more actuatable lighting modules, arrays of light-emitting elements, optical components, sensors, or interface components. The lighting assembly may be configured as a unitary structure or as a collection of interconnected components arranged to illuminate a procedural surface or other target area. In some embodiments, a lighting assembly may be extendable, retractable, and/or angularly adjustable to direct illumination toward selected regions with enhanced precision.

In some embodiments, the lighting assembly may define an oval, circular, square, rectangular, or other perimeter and may be dimensioned to extend beyond the perimeter of a patient support surface or other target area. In some embodiments, the intelligent lighting system may include multiple arrays of light-emitting elements and a plurality of actuatable lighting modules. For example, in an embodiment, the intelligent lighting system may include a plurality of actuatable lighting modules arranged at regular or irregular intervals about the perimeter of the lighting assembly or at other locations. Between adjacent actuatable lighting modules, at least one or a plurality of light-emitting elements may be disposed to provide additional lighting. In some embodiments, each actuatable lighting module may include one or more internal illumination sources and a sensor. The illumination sources may be one of an LED, halogen, fluorescent, incandescent, or other light source.

The mounted configuration may permit the lighting assembly to be positioned above the procedural surface while reducing floor obstructions and enabling controlled articulation, rotation, sensing, power delivery, and distribution of light relative to the target region.

In some embodiments, a “sensing component” or a “sensor” may include one or more cameras, depth sensors, imaging sensors, position sensors, occupancy sensors, ambient light sensors, temperature sensors, optical sensors, infrared cameras, visible-light cameras, wireless signal sensors, magnetic sensors, or other sensors configured to detect conditions associated with the procedural surface, the surrounding environment, the lighting assembly, a region of interest, a target area including a patient, or a user. In some embodiments, an optical sensor may be capable of recording video or still images from the procedure which may be stored on a local or external memory module for later use.

In some embodiments, the “control electronics” may include one or more processors, controllers, driver circuits, communication interfaces, memory devices, power-control circuits, or other electronic components configured to coordinate operation of the intelligent lighting system, receive sensor data, user commands, or stored operational parameters, execute control logic, and control operation of the lighting assembly. In some embodiments, the control electronics may employ machine vision, artificial intelligence, or machine learning techniques to identify personnel, detect movement, track a region of interest, and/or predict future illumination requirements and adjust illumination accordingly.

As used herein, a “power component” may include one or more power supplies, transformers, batteries, power conditioning circuits, charging components, electrical connectors, or power distribution components configured to provide operating power to the lighting assembly, the sensing components, and the control electronics.

Based on real-time analysis, the intelligent lighting system may autonomously adjust one or more articulating or extendable lighting modules to minimize shadow formation and maintain consistent illumination over regions of interest. These adjustments may occur dynamically and seamlessly, without disrupting workflow or drawing attention from the procedural team based on learned behaviors and trained artificial intelligence (AI) or machine learning systems.

The intelligent lighting system may include one or more audio input devices, e.g., microphones or smart speakers, configured to activate a control system upon receiving a designated input followed by a control instruction, e.g., to power on the lighting modules. In some embodiments, the control electronics may further control repositioning, focus, extension, retraction, beam direction, image capture, diagnostic operation, or other functions of one or more lighting modules, and may respond to voice commands, marker-based inputs, or other user control inputs to support hands-free operation. Example voice commands include “turn lights on,” “increase light intensity,” “decrease light intensity,” “switch to assistant control,” “focus lights on the liver,” “focus lights on the incision site,” and “take picture.”

In some embodiments, one or more tracking features may be positioned within the procedural field to assist with calibration and/or use of the system. The one or more tracking features may be incorporated on or into clothing, hand coverings, instruments, or other articles positioned within the procedural field. The tracking features may include pins, clips, appliqués, printed marks, woven marks, embedded markers, or other detectable features having unique patterns that may be detected by one or more sensors of the lighting assembly. Such tracking features may allow the system to properly track individuals or items in the system, for example, during a training session or during a procedure.

In some embodiments, a primary operator may wear one or more wearable articles, such as hand coverings or gloves, including infrared-reflective or patterned markers detectable by the system's optical sensors. The intelligent lighting system may be configured to recognize distinct marker patterns associated with different users and, for example, in response to a control input, transfer control functionality to a secondary user wearing a uniquely coded set of hand coverings, wherein the control input may include input received via the one or more audio input devices. Alternatively, in some embodiments, the system may be trained to automatically follow the primary user without the need for tracking features until the system is instructed otherwise. For example, the primary user may direct the system, via one or more control inputs, to follow a secondary user for a period.

In certain embodiments, during the course of a procedure, the intelligent lighting system may employ machine learning algorithms to analyze procedural patterns and infer workflow progression. For example, the system may increase or decrease the intensity or color of emitted light based on procedural requirements. Based on real-time data inputs, the system may generate alerts indicating potential intraoperative complications, such as indications of vascular disruption or bleeding, prior to case completion. Procedural footage and sensor data may be recorded, anonymized in accordance with applicable laws (e.g., HIPAA), and made accessible to the procedural team for post-procedure review. In some embodiments, the system may interface with a remote or cloud-based platform to facilitate case sharing, peer review, and dissemination of procedural best practices among authorized users.

In some embodiments, system operation may include an initial calibration in which the system divides a procedural table, which may include one or more markers, into distinct quadrants, such as quadrants A, B, C and D, each of which may be further subdivided into numbered zones, such as A1, A2, A3, etc. The quadrants and zones may serve as reference locations for training of the system, thereby enabling the system to recognize features, such as organs and other anatomical structures, for targeted illumination. In some embodiments, the intelligent lighting system may respond to inputs from a computer vision system used in combination with one or more infrared markers, such as an infrared-marked glove, and may generate autonomous control outputs to telescopic and non-telescopic light sources. During use, the system may continue to learn user-specific preferences and/or characteristics of a procedure or procedural environment.

In some embodiments, the system may support machine learning and training by assigning labels to regions of the procedural table. For example, the procedural table may be divided into four quadrants, with each quadrant including multiple nodes and corresponding lighting foci, and with one or more sub-cluster foci providing more refined node identification, such as quadrant C, zone C1, clusters 1-4. The clusters may permit more refined anatomy identification and alignment of anatomy with nodal references, thereby enabling user control inputs, including voice commands and marker-based inputs, and corresponding autonomous light adjustments. For example, the system may responsively direct illumination to a selected region associated with the operating room table 6, such as quadrant D.

As disclosed herein, an intelligent lighting system may be configured to provide adaptive illumination within an environment, such as a surgical or other procedural environment. The intelligent lighting system may include one or more lighting assemblies, sensing components, control electronics, and power components configured to cooperate to illuminate a procedural surface or other target area. In some embodiments, the sensing components may detect conditions associated with the target area, the surrounding environment, the lighting assembly, a region of interest, or a user, and the control electronics may responsively adjust one or more illumination characteristics based on detected inputs, learned patterns, stored operational parameters, or user commands. In this manner, the disclosed system may reduce reliance on manual lighting adjustment while supporting accurate, stable, and responsive illumination.

In some embodiments, the intelligent lighting system may be implemented in a mobile floor-supported configuration or a ceiling-mounted configuration. A support assembly may position one or more lighting assemblies relative to a target area and may include articulated members, adjustable mounts, pivoting couplings, translational components, actuatable joints, or other positioning structures configured to permit controlled rotational, angular, translational, vertical, lateral, or other movement of the lighting assembly. In some embodiments, the lighting assembly may include one or more light sources, actuatable lighting modules, arrays of light-emitting elements, optical components, sensors, or interface components arranged as a unitary structure or as interconnected components. The lighting assembly may extend about, surround, or partially encircle a procedural surface and may define an oval, circular, square, rectangular, or other perimeter.

In some embodiments, the sensing components may include one or more cameras, depth sensors, imaging sensors, optical sensors, infrared cameras, visible-light cameras, position sensors, occupancy sensors, ambient light sensors, temperature sensors, wireless signal sensors, magnetic sensors, or other sensors. In some embodiments, the control electronics may include one or more processors, controllers, driver circuits, communication interfaces, memory devices, power-control circuits, or other electronic components configured to receive sensed data, execute control logic, and control operation of the lighting assembly. The control electronics may employ machine vision, artificial intelligence, or machine learning techniques to identify personnel, detect movement, track a region of interest, predict illumination requirements, and adjust illumination accordingly. In some embodiments, the control electronics may further control repositioning, focus, extension, retraction, beam direction, image capture, or diagnostic operation, and may respond to voice commands, marker-based inputs, or other user inputs to support hands-free operation.

In some embodiments, one or more sensors of the lighting assembly include a computer vision camera configured to capture image data of a user's hand within the procedural environment. The control electronics may be configured to identify one or more predefined hand movements or gestures from the image data and to associate the one or more predefined hand movements or gestures with corresponding lighting commands. For example, a detected gesture may cause the control electronics to move the lighting assembly, redirect illumination toward a different region of interest, change an angle of emitted light, adjust focus, increase or decrease light intensity, or maintain illumination at a selected location. In some embodiments, gesture-based control may be used alone or in combination with marker-based tracking, voice-based control, and autonomous tracking of a region of interest.

In some embodiments, the intelligent lighting system may further obtain image data, spectral data, thermal data, or other sensed data from a target region and may process such data to perform diagnostic functions. For example, the disclosed system may assess tissue characteristics, detect abnormalities, identify regions of interest, monitor procedural conditions, generate alerts, capture procedural images, or record procedural footage and sensor data for later review. The disclosed systems and methods may be used in medical, surgical, or other procedural environments, as well as in industrial maintenance settings, construction sites, inspection operations, laboratory environments, workshop or hobby environments, educational settings, manufacturing facilities, automotive repair environments, and other commercial, residential, or technical applications in which adaptive illumination, sensing, positioning, monitoring, imaging, or diagnostic functionality is desirable.

Features from any of the embodiments described herein may be used in combination with one another in accordance with the general principles described herein. These and other embodiments, features, and advantages will be more fully understood upon reading the detailed description in conjunction with the accompanying drawings and claims.

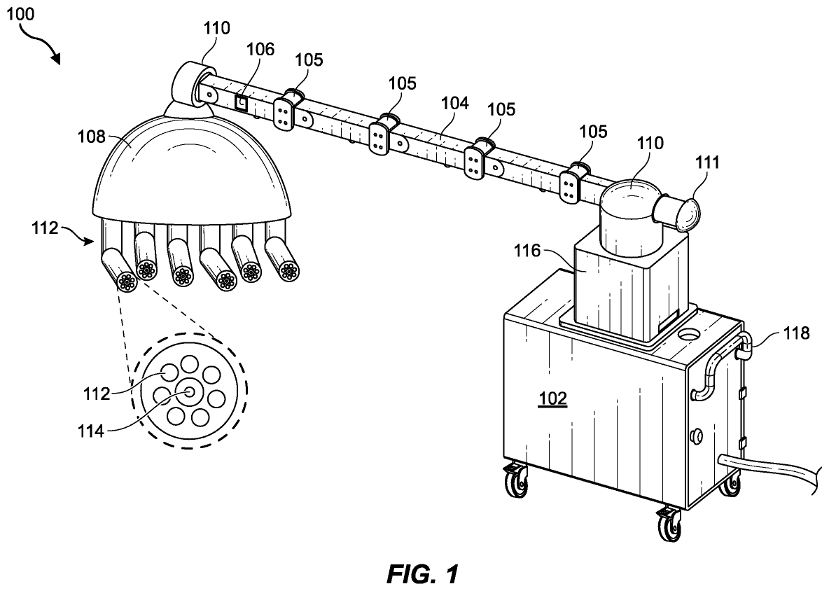

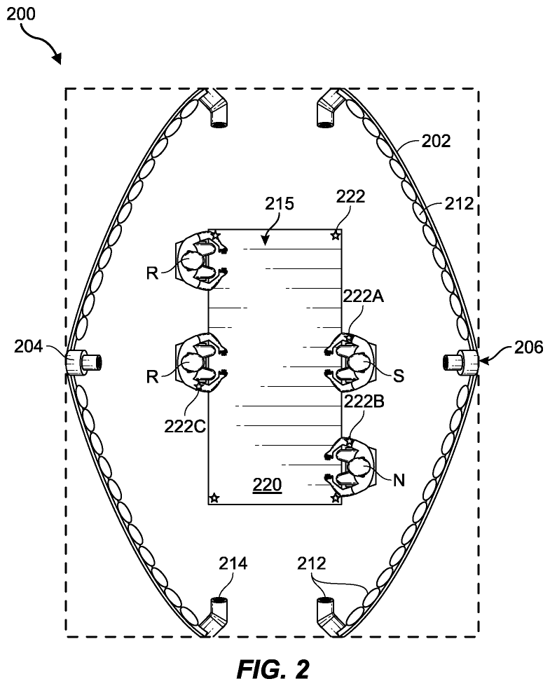

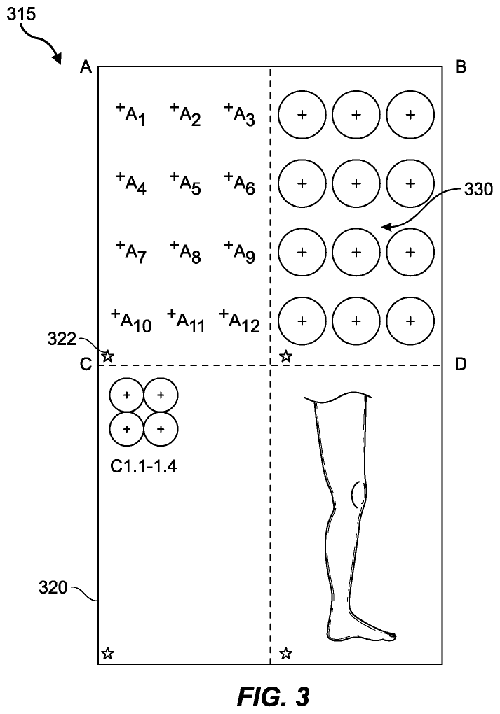

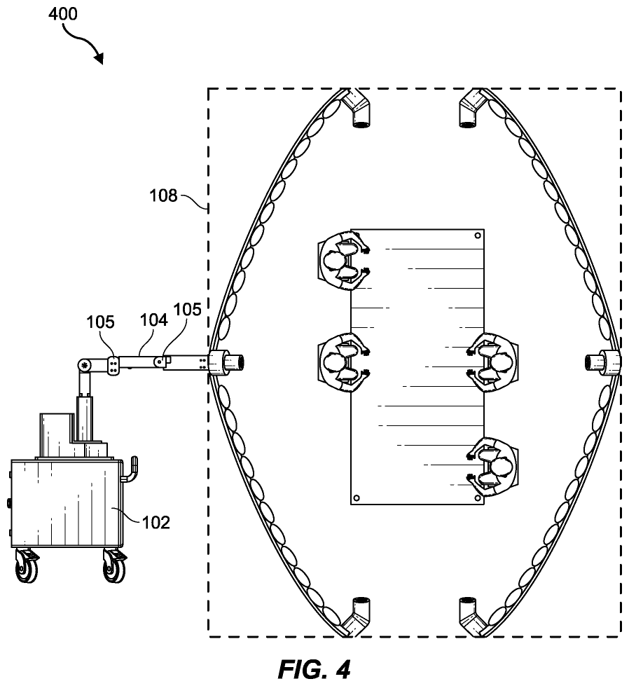

The following will provide, with reference to FIGS. 1-4, detailed descriptions of intelligent lighting systems and example methods of operation. The description associated with FIG. 1 relates to an example intelligent lighting system in a mobile floor-supported configuration including a movable base structure, an adjustable arm, and a lighting assembly having one or more light sources and one or more integrated sensors. The description associated with FIG. 2 describes a further example intelligent lighting system in a mounted configuration including a support truss, a support assembly, and a lighting assembly configured to interact with infrared markers, infrared-marked gloves, and a grid-identified procedural table. The description associated with FIG. 3 describes partitioning a procedural environment into zones to facilitate detection, tracking, and responsive control of illumination within selected regions of the procedural environment. The description associated with FIG. 4 describes a further example intelligent lighting system similar to the intelligent lighting system of FIG. 2, but in a mobile floor-supported configuration including a movable base structure instead of a mounted configuration.

Turning to FIG. 1, an example intelligent lighting system is shown. In some embodiments, intelligent lighting system 100 may include a movable base structure 102 configured to support and position the system within a procedural environment. Base structure 102 may be free-standing and, in some embodiments, may include wheels or another mobile support arrangement to permit repositioning of intelligent lighting system 100 relative to a target area. In some embodiments, the base structure 102 may include one or more power cords, data cords, or other connection elements configured to couple the system to a power source, a data source, or both.

In some embodiments, intelligent lighting system 100 may further include an adjustable arm 104 extending from base structure 102. Adjustable arm 104 may include one or more articulated segments, joints, servo-driven couplings, or other actuatable positioning components configured to provide controlled movement of the distal portion of adjustable arm 104 relative to the target area.

In some embodiments, the adjustable arm 104 may be length-adjustable via actuation of a telescoping linkage. In some embodiments, adjustable arm 104 may provide multiple degrees of freedom, thereby permitting rotational, angular, vertical, lateral, and/or translational movement. One or more motion, position, or proximity sensors 106 may be positioned along adjustable arm 104 and may be configured to detect nearby objects or personnel to reduce or avoid unintended contact during operation. In some embodiments, the position of the adjustable arm 104 may be adjusted using pneumatic, hydraulic, and/or electrical actuators 105. In some embodiments, the adjustable arm 104 may be configured to retract when not in use.

A lighting assembly 108 may be supported at a distal end of adjustable arm 104. In some embodiments, lighting assembly 108 may be coupled to adjustable arm 104 by an actuatable joint 110, such as a rotatable joint, pivot joint, ball joint, universal joint, or other multidirectional coupling configured to permit selective positioning of lighting assembly 108. In some embodiments, a counterweight 111 may be coupled to adjustable arm 104 to offset at least a portion of the load associated with movement of lighting assembly 108.

Lighting assembly 108 may include one or more light sources 112 configured to illuminate a procedural field or other target area. In some embodiments, lighting assembly 108 may further include one or more sensors 114 configured to detect conditions within the procedural environment. Sensors 114 may include, for example, one or more visible-light cameras, infrared cameras, depth sensors, optical sensors, or other imaging devices. In some embodiments, the one or more light sources 112 may be arranged around or adjacent to the one or more sensors 114 such that lighting assembly 108 provides both illumination and sensing functionality from a common assembly.

Intelligent lighting system 100 may further include control electronics 116 configured to communicate with proximity sensors 106, actuatable joint(s) 110, light sources 112 and/or sensors 114. In some embodiments, control electronics 116 may be supported by base structure 102 and may include one or more processors, controllers, memory devices, communication interfaces, or driver circuits configured to receive sensed data and generate control outputs for repositioning lighting assembly 108 and adjusting illumination. In some embodiments, control electronics 116 may cause lighting assembly 108 to direct illumination from a first location to a second location based on detected movement, marker-based inputs, voice commands, learned procedural patterns, or combinations thereof.

In some embodiments, intelligent lighting system 100 may include one or more user interface components 118, such as a display, touchscreen, control panel, microphone, speaker, or other input/output device. The one or more user interface components 118 may permit a user to provide commands to intelligent lighting system 100 and/or receive operating information from intelligent lighting system 100. Example commands may include commands to activate the system, adjust illumination intensity, alter illumination color, reposition lighting assembly 108, capture image data, or transfer control between users.

In some embodiments, prior to a procedure and during use, the base structure 102 may be positioned at a periphery of a procedural space to reduce interference with the procedural field and maintain access for the procedural team. In some embodiments, the base structure configuration may be adjustable and operable through one or more remote input methods, including one or more audio input devices configured to receive control inputs, such as voice inputs.

During operation, sensors 114 may detect one or more conditions associated with the target area, one or more users, one or more instruments, and/or the surrounding environment. Control electronics 116 may process the detected information and responsively control operation of lighting assembly 108 such that illumination follows a region of interest, reduces shadow formation, and/or adapts to changing procedural conditions. In some embodiments, marker-based tracking and/or voice-based control may permit substantially hands-free operation of intelligent lighting system 100.

Referring to FIG. 2, a further example intelligent lighting system is shown in a mounted configuration. In some embodiments, intelligent lighting system 200 may include a support truss 202 configured to support the system above a procedural environment. Support truss 202 may be secured to or suspended from a ceiling or other elevated support and may carry a support assembly 204 configured to position a lighting assembly 206 relative to a procedural field.

In some embodiments, support assembly 204 may include one or more rigid members, articulated members, telescopic members, pivoting couplings, translational components, and/or other positioning structures configured to permit controlled movement of lighting assembly 206. For example, support assembly 204 may provide vertical, lateral, rotational, angular, and/or translational adjustment of lighting assembly 206 relative to a target area. In some embodiments, support assembly 204 and/or lighting assembly 206 may further include adjustable position control configured to responsively reposition lighting assembly 206 during use.

Lighting assembly 206 may include one or more light sources 212 configured to illuminate a procedural field or other target area. In some embodiments, the one or more light sources 212 may include telescopic and/or non-telescopic lighting modules, and one or more of the light sources 212 may be actuatable to angulate, focus, and/or otherwise direct emitted illumination toward a selected location. In some embodiments, lighting assembly 206 may further include one or more sensors 214 integrated into the lighting assembly 206. Sensors 214 may include, for example, a visible-light camera, an infrared camera, and/or one or more additional optical or imaging sensors configured to detect features within the procedural environment. In some embodiments, one or more light sources 212 may be arranged around or adjacent to sensors 214 such that lighting assembly 206 provides both illumination and sensing functionality from a common overhead assembly.

In some embodiments, intelligent lighting system 200 may further include control electronics (not shown) configured to communicate with lighting assembly 206, light sources 212, and/or sensors 214. Control electronics may include one or more processors, controllers, memory devices, communication interfaces, and/or driver circuits configured to receive sensed data and generate control outputs for repositioning lighting assembly 206 and adjusting illumination.

The procedural environment 215 may include one or more infrared markers positioned within or about the procedural field. In some embodiments, the one or more infrared markers 222 may be positioned at spaced locations to assist with calibration and positional registration of intelligent lighting system 200. In some embodiments, the procedural environment may further include one or more infrared-marked gloves associated with respective members of the procedural team. For example, infrared-marker gloves 222A, 222B, and 222C may include different infrared-reflective and/or patterned features configured to permit intelligent lighting system 200 to distinguish between different users, such as a surgeon(S), nurse (N), or resident (R).

In some embodiments, the procedural environment 215 may further include a procedural table 220 having a grid-identified positional reference, such as IR markers 222. Positional reference arrangement may include quadrants, zones, nodes, sub-clusters, and/or other location identifiers configured to assist with training, calibration, and targeted illumination. Sensors 214 in lighting assembly 206 may detect infrared markers 222 and/or infrared-marked gloves 222A, 222B, 222C to determine a location of interest within the procedural environment.

During operation, control electronics may process image data and other sensed data obtained by sensors 214 and may responsively control adjustable position control and/or light sources 212 such that illumination is directed to a selected quadrant, zone, node, or other region of interest within procedural environment 215. In some embodiments, intelligent lighting system 200 may use computer vision processing in combination with infrared detection to track hand location, instrument location, tissue location, shadow formation, and/or movement within the procedural field. Based on such detected conditions, intelligent lighting system 200 may automatically move, focus, angulate, or otherwise control lighting assembly 206 to maintain illumination over the desired region while reducing shadow formation and supporting substantially hands-free operation.

Referring to FIG. 3, an example partitioned procedural environment is shown. In some embodiments, procedural environment 315 may include a procedural table 320 or other target area configured to support a patient, specimen, workpiece, or other subject of interest during a procedure. In some embodiments, procedural table 320 may define a procedural field 330 within which an intelligent lighting system may monitor activity and responsively direct illumination.

In some embodiments, procedural field 330 may include one or more positional reference markers 322 configured to partition procedural environment 315 into a plurality of defined regions. Positional reference markers 322 may include a grid, overlay, projected reference pattern, stored virtual mapping, or other reference structure capable of identifying locations within procedural field 330. In some embodiments, positional reference markers 322 may divide procedural field 330 into quadrants, e.g., A, B, C, D.

In some embodiments, each quadrant may be further subdivided into a plurality of zones, e.g., A1, A2, A3, etc., that permit more precise localization within procedural field 330.In some embodiments, one or more of the zones (e.g., zone C1) may further include nodes or sub-clusters, e.g., C1.1-C1.4, configured to provide increasingly refined positional identification for a region of interest. By way of example, a location within a quadrant may be identified at the quadrant level, the zone level, the node level, and/or the sub-cluster level to permit targeted illumination with increased precision.

In some embodiments, positional reference markers 322 may be used during an initial calibration process of the intelligent lighting system. For example, the intelligent lighting system may identify quadrants, zones, nodes, and/or sub-clusters and associate those regions with corresponding lighting positions, lighting foci, or learned procedural references. In some embodiments, the quadrants and associated subdivisions may serve as reference locations for system training, thereby enabling the intelligent lighting system to recognize anatomical structures, instruments, workflow locations, or other features of interest and to responsively direct illumination to corresponding regions.

During operation, the intelligent lighting system may process sensed data associated with procedural environment 315 and may responsively direct illumination to a selected quadrant, zone, node, or sub-cluster. In some embodiments, the system may be configured to restrict operation to a selected quadrant for a given procedure, thereby reducing unnecessary tracking outside the relevant portion of procedural environment 315. In some embodiments, the system may respond to computer vision inputs, infrared marker detection, marker-based inputs, voice commands, learned procedural patterns, or combinations thereof to move, focus, angulate, or otherwise control one or more telescopic and/or non-telescopic light sources.

In some embodiments, the partitioning of procedural environment 315 shown in FIG. 3 may permit the intelligent lighting system to provide more accurate detection, tracking, and illumination control within selected regions of procedural environment 315. By assigning labels to quadrants, zones, nodes, and sub-clusters, the system may support training, user-specific preferences, anatomy identification, and finer-grained targeting of illumination while reducing reliance on manual repositioning during a procedure.

Turning to FIG. 4, illustrated is a further example intelligent lighting system 400 similar to the intelligent lighting system 200 of FIG. 2, but in a mobile floor-supported configuration including a movable base structure 102, instead of a mounted configuration, supporting a light assembly 108.

### EXAMPLE EMBODIMENTS

Example 1: A lighting system includes a support assembly, a lighting assembly movably coupled to the support assembly, the lighting assembly including one or more light sources and one or more sensors configured to generate sensor data associated with an environment, and a control system operatively coupled to the support assembly and the lighting assembly, the control system being configured to receive the sensor data from the one or more sensors, determine, based on the sensor data, a location of activity or a region of interest within the environment; and automatically control at least one of the support assembly or the lighting assembly to adjust at least one of a position of the lighting assembly, an orientation of the lighting assembly, or an illumination characteristic of the one or more light sources such that illumination is directed in response to the location of activity or the region of interest.

Example 2: The lighting system of Example 1, where the support assembly includes a support structure selected from a floor-supported base, a wall mount, a ceiling mount, and a table-mounted support.

Example 3: The lighting system of any of Examples 1 and 2, where the support assembly includes one or more actuators configured to move the lighting assembly in response to control signals generated by the control system.

Example 4: The lighting system of any of Examples 1-3, where the support assembly includes an arm supporting the lighting assembly.

Example 5: The lighting system of Example 4, where the arm includes one or more articulated joints configured to permit repositioning of the lighting assembly relative to the environment.

Example 6: The lighting system of any of Examples 4 and 5, where the arm includes a telescoping portion configured to vary a distance between the lighting assembly and an object in the environment.

Example 7: The lighting system of any of Examples 4-6, where a position sensor is disposed on the arm and configured to generate position data indicative of a position of the arm.

Example 8: The lighting system of any of Examples 4-7, where a proximity sensor is disposed on the arm and configured to generate sensor data indicative of an object near the arm, and the control system is configured to control movement of the support assembly based on the sensor data to avoid collision between the arm and the object.

Example 9: The lighting system of any of Examples 1-8, where the one or more sensors of the lighting assembly include a visible-light camera or an infrared camera.

Example 10: The lighting system of any of Examples 1-9, where the lighting assembly includes one or more actuatable lighting modules configured to angulate or focus emitted illumination toward a selected location in the environment.

Example 11: A lighting system includes a support assembly, a lighting assembly movably coupled to the support assembly, the lighting assembly including one or more light sources and one or more sensors configured to generate sensor data associated with a target area in an environment, and a control system operatively coupled to the support assembly and the lighting assembly, where one or more tracking features are disposed in or about the target area, and where the control system is configured to receive the sensor data from the one or more sensors, detect, based on the sensor data, at least one of the one or more tracking features or a selected region within the target area, determine, based on the detected at least one of the one or more tracking features or the selected region, a region of interest within the target area, and automatically control at least one of the support assembly or the lighting assembly to adjust at least one of a position of the lighting assembly, an orientation of the lighting assembly, or an illumination characteristic of the one or more light sources such that illumination is directed to the region of interest.

Example 12: The lighting system of Example 11, where the one or more tracking features include one or more infrared markers positioned within or about the target area.

Example 13: The lighting system of any of Examples 11 and 12, where the one or more tracking features are disposed on one or more wearable articles configured to be worn by a user.

Example 14: The lighting system of any of Examples 11-13, where the control system is configured to distinguish between different users based on different marker patterns associated with respective ones of the wearable articles.

Example 15: The lighting system of any of Examples 11-14, where the control system is configured to direct illumination to a selected quadrant, zone, node, or cluster of the target area.

Example 16: A method includes providing a lighting system including a support assembly, a lighting assembly movably coupled to the support assembly, the lighting assembly having one or more light sources and one or more sensors, and a control system operatively coupled to the support assembly and the lighting assembly, receiving, by the control system, sensor data from the one or more sensors, detecting, by the control system and based on the sensor data, one or more tracking features disposed in or about a target area in an environment, identifying, by the control system and based on the sensor data and the detected one or more tracking features, a region of interest within the target area, and automatically controlling, by the control system, at least one of the support assembly or the lighting assembly to adjust at least one of a position of the lighting assembly, an orientation of the lighting assembly, or an illumination characteristic of the one or more light sources such that illumination is directed to the region of interest.

Example 17: The method of Example 16, where the one or more tracking features are disposed on wearable articles worn by respective users, the method further including distinguishing, by the control system, between the respective users based on different patterns of the one or more tracking features and automatically directing the illumination to follow a selected one of the respective users.

Example 18: The method of any of Examples 16 and 17, further including processing, by the control system, the sensor data using computer vision, artificial intelligence, or machine learning to detect movement, track the region of interest, predict a future illumination requirement, and automatically adjust the illumination characteristic based on the predicted future illumination requirement.

Example 19: The method of any of Examples 16-18, further including detecting, by the control system, shadow formation within the target area and automatically controlling at least one of the support assembly or the lighting assembly to reduce the shadow formation while maintaining illumination over the region of interest.

Example 20: The method of any of Examples 16-19, further including learning, by the control system during use of the lighting system, one or more user-specific preferences or procedural patterns and automatically adjusting at least one of the position of the lighting assembly, the orientation of the lighting assembly, or the illumination characteristic of the one or more light sources based on the learned user-specific preferences or procedural patterns.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

It will be understood that when an element such as a layer or a region is referred to as being formed on, deposited on, or disposed “on” or “over” another element, it may be located directly on at least a portion of the other element, or one or more intervening elements may also be present. In contrast, when an element is referred to as being “directly on” or “directly over” another element, it may be located on at least a portion of the other element, with no intervening elements present.

As used herein, the term “approximately” in reference to a particular numeric value or range of values may, in certain embodiments, mean and include the stated value as well as all values within 10% of the stated value. Thus, by way of example, reference to the numeric value “50” as “approximately 50” may, in certain embodiments, include values equal to 50±5, i.e., values within the range 45 to 55.

As used herein, the term “substantially” in reference to a given parameter, property, or condition may mean and include to a degree that one of ordinary skill in the art would understand that the given parameter, property, or condition is met with a small degree of variance, such as within acceptable manufacturing tolerances. By way of example, depending on the particular parameter, property, or condition that is substantially met, the parameter, property, or condition may be at least approximately 90% met, at least approximately 95% met, or even at least approximately 99% met.

While various features, elements or steps of particular embodiments may be disclosed using the transitional phrase “comprising,” it is to be understood that alternative embodiments, including those that may be described using the transitional phrases “consisting of” or “consisting essentially of,” are implied. Thus, for example, implied alternative embodiments to a light source that comprises or includes a light emitting diode include embodiments where a light source consists essentially of a light emitting diode and embodiments where a light source consists of a light emitting diode.

What is claimed is:

1. A lighting system, comprising:
a support assembly;
a lighting assembly movably coupled to the support assembly, the lighting assembly comprising one or more light sources and one or more sensors configured to generate sensor data associated with an environment; and
a control system operatively coupled to the support assembly and the lighting assembly, the control system being configured to:
receive the sensor data from the one or more sensors;
determine, based on the sensor data, a location of activity or a region of interest within the environment; and
automatically control at least one of the support assembly or the lighting assembly to adjust at least one of a position of the lighting assembly, an orientation of the lighting assembly, or an illumination characteristic of the one or more light sources such that illumination is directed in response to the location of activity or the region of interest.

2. The lighting system of claim 1, wherein the support assembly comprises a support structure selected from the group consisting of a floor-supported base, a wall mount, a ceiling mount, and a table-mounted support.

3. The lighting system of claim 1, wherein the support assembly comprises one or more actuators configured to move the lighting assembly in response to control signals generated by the control system.

4. The lighting system of claim 1, wherein the support assembly comprises an arm supporting the lighting assembly.

5. The lighting system of claim 4, wherein the arm comprises one or more articulated joints configured to permit repositioning of the lighting assembly relative to the environment.

6. The lighting system of claim 4, wherein the arm comprises a telescoping portion configured to vary a distance between the lighting assembly and an object in the environment.

7. The lighting system of claim 4, wherein a position sensor is disposed on the arm and configured to generate position data indicative of a position of the arm.

8. The lighting system of claim 4, wherein a proximity sensor is disposed on the arm and configured to generate sensor data indicative of an object near the arm, and the control system is configured to control movement of the support assembly based on the sensor data to avoid collision between the arm and the object.

9. The lighting system of claim 1, wherein the one or more sensors of the lighting assembly comprise a visible-light camera or an infrared camera.

10. The lighting system of claim 1, wherein the lighting assembly comprises one or more actuatable lighting modules configured to angulate or focus emitted illumination toward a selected location in the environment.

11. A lighting system, comprising:
a support assembly;
a lighting assembly movably coupled to the support assembly, the lighting assembly comprising one or more light sources and one or more sensors configured to generate sensor data associated with a target area in an environment; and
a control system operatively coupled to the support assembly and the lighting assembly,
wherein one or more tracking features are disposed in or about the target area, and
wherein the control system is configured to:
receive the sensor data from the one or more sensors;
detect, based on the sensor data, at least one of the one or more tracking features or a selected region within the target area;
determine, based on the detected at least one of the one or more tracking features or the selected region, a region of interest within the target area; and
automatically control at least one of the support assembly or the lighting assembly to adjust at least one of a position of the lighting assembly, an orientation of the lighting assembly, or an illumination characteristic of the one or more light sources such that illumination is directed to the region of interest.

12. The lighting system of claim 11, wherein the one or more tracking features comprise one or more infrared markers positioned within or about the target area.

13. The lighting system of claim 11, wherein the one or more tracking features are disposed on one or more wearable articles configured to be worn by a user.

14. The lighting system of claim 13, wherein the control system is configured to distinguish between different users based on different marker patterns associated with respective ones of the wearable articles.

15. The lighting system of claim 11, wherein the control system is configured to direct illumination to a selected quadrant, zone, node, or cluster of the target area.

16. A method comprising:
providing a lighting system including a support assembly, a lighting assembly movably coupled to the support assembly, the lighting assembly comprising one or more light sources and one or more sensors, and a control system operatively coupled to the support assembly and the lighting assembly;
receiving, by the control system, sensor data from the one or more sensors;
detecting, by the control system and based on the sensor data, one or more tracking features disposed in or about a target area in an environment;
identifying, by the control system and based on the sensor data and the detected one or more tracking features, a region of interest within the target area; and
automatically controlling, by the control system, at least one of the support assembly or the lighting assembly to adjust at least one of a position of the lighting assembly, an orientation of the lighting assembly, or an illumination characteristic of the one or more light sources such that illumination is directed to the region of interest.

17. The method of claim 16, wherein the one or more tracking features are disposed on wearable articles worn by respective users, the method further comprising distinguishing, by the control system, between the respective users based on different patterns of the one or more tracking features and automatically directing the illumination to follow a selected one of the respective users.

18. The method of claim 16, further comprising processing, by the control system, the sensor data using computer vision, artificial intelligence, or machine learning to detect movement, track the region of interest, predict a future illumination requirement, and automatically adjust the illumination characteristic based on the predicted future illumination requirement.

19. The method of claim 16, further comprising detecting, by the control system, shadow formation within the target area and automatically controlling at least one of the support assembly or the lighting assembly to reduce the shadow formation while maintaining illumination over the region of interest.

20. The method of claim 16, further comprising learning, by the control system during use of the lighting system, one or more user-specific preferences or procedural patterns and automatically adjusting at least one of the position of the lighting assembly, the orientation of the lighting assembly, or the illumination characteristic of the one or more light sources based on the learned user-specific preferences or procedural patterns.
